(19) United States
(12) Patent Application Publication
(10) Pub. No.: US 2010/0167398 A1
(43) Pub. Date: Jul. 1, 2010

(54) METHOD OF CULTURING PLURIPOTENT STEM CELLS USING EXTRACELLULAR MATRIX FROM FETAL MEMBRANE-DERIVED CELLS

(75) Inventors: Yoshiki SASAI, Hyogo (JP); Tomoko NAGASE, Hyogo (JP); Morio UENO, Hyogo (JP); Yonehiro KANEMURA, Osaka (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: RIKEN, Wako-shi (JP)

(21) Appl. No.: 12/646,156

(22) Filed: Dec. 23, 2009

(30) Foreign Application Priority Data
Dec. 24, 2008 (JP) 2008-328339
Aug. 27, 2009 (JP) 2009-197239

(51) Int. Cl. C12N 5/071; C12N 5/02; C12M 1/00

(52) U.S. Cl. 435/366; 435/373; 435/289.1

(57) ABSTRACT

Provided are a method of culturing pluripotent stem cells in the presence of a decidua-derived cell or an extracellular matrix derived from the cell, that enables safe and efficient maintenance culture and derivation of pluripotent stem cells; a culture agent for pluripotent stem cells, that comprises a decidua-derived cell or an extracellular matrix derived from the cell; and other means for developing or performing the method.

### TECHNICAL FIELD

The present invention relates to a novel method of culturing pluripotent stem cells. More specifically, the present invention relates to a method of culturing pluripotent stem cells using decidua-derived cells, particularly a decidua-derived mesenchymal cell, or an extracellular matrix obtained from the cell; a culture agent for pluripotent stem cells, comprising a decidua-derived mesenchymal cell or an extracellular matrix derived from the cell; a container for culturing pluripotent stem cells, coated with an extracellular matrix from decidua-derived mesenchymal cells, and the like.

### BACKGROUND ART

There are high expectations for human pluripotent stem cells such as human embryonic ES (hES) cells and human induced pluripotent stem (hiPS) cells as excellent source materials for cell therapy for intractable diseases. Derivatization and maintenance culture of hES cells are routinely conducted at many laboratories, and the cells cultured are induced to differentiate into medically useful cell types, for example, dopaminergic neurons, cardiomyocytes, islet cells and the like.

Conventionally, human pluripotent stem cells such as human ES cells and iPS cells have been co-cultured with feeder cells of animal (particularly mouse) origin [e.g., mouse embryonic fibroblasts (MEF)] to achieve maintenance culture and establish cell lines. Because a culturing method employing any ingredient of animal (e.g., mouse) origin involves risks for infection and antigenicity as with heterotransplantation, however, a safe alternative method is required for use in medical settings.

There are two approaches known to serve as MEF substitutes. One is the use of human feeder cells. Maintenance culture of hES cells using a primary culture of foreskin fibroblasts and the like has been reported (Hovatta O, Mikkola M, Gertow K et al., A culture system using human foreskin fibroblasts as feeder cells allows production of human embryonic stem cells. Hum Reprod 2003; 18:1404-1409). Activities that enable maintenance culture of human pluripotent stem cells have also been reported in various other human cell types [adult oviduct epithelial cells, fetal skin fibroblasts (Richards M, Fong C Y, Chan W K et al., Human feeders support prolonged undifferentiated growth of human inner cell masses and embryonic stem cells. Nat Biotechnol 2002; 20:933-936), marrow mesenchymal cells (Cheng L, Hammond H, Ye Z et al., Human adult marrow cells support prolonged expansion of human embryonic stem cells in culture. Stem Cells 2003; 21:131-142), and adult skin fibroblasts (Richards M, Tan S, Fong C Y et al., Comparative evaluation of various human feeders for prolonged undifferentiated growth of human embryonic stem cells. Stem Cells 2003; 21:546-556)]. However, tissues of human origin are difficult to obtain, and are not easy to use routinely. In addition to the necessity for the obtainment of appropriate informed consent from the donor patient, the amount of tissue obtained is usually small, and the number of passages is often limited. Furthermore, the maintenance culture activity of human feeder cells often varies among different batches, so it is difficult to obtain the activity constantly with high reproducibility.

The other MEF substitute is the use of extracellular matrix as the culture substrate (JP-A-2006-59). Because matrix substrates are cell-free, the problem of contamination with feeder cells in hES cells and the like in culture can be avoided. Additionally, matrix substrates are usually stable. Produced from a mouse tumor cell line (EHS sarcoma), Matrigel is the most commonly used extracellular matrix, exhibiting potent maintenance culture activity for hES cells and the like in culture (Xu C, Inokuma M S, Denham J et al., Feeder-free growth of undifferentiated human embryonic stem cells. Nat Biotechnol 2001; 19:971-974). The extracellular matrix of MEF has also been reported to have similar maintenance culture activity (Klimanskaya I, Chung Y, Meisner L et al., Human embryonic stem cells derived without feeder cells. Lancet 2005; 365:1636-1641). However, these matrixes are of animal origin, and cannot be free from an ingredient of heterologous animal origin. Therefore, this case involves a risk in medical application as with the use of MEF.

### SUMMARY OF THE INVENTION

### Problems to Be Solved by the Invention

It is an object of the present invention to provide a method that enables safe and efficient maintenance culture and establishment of human pluripotent stem cells, and various means for developing or performing the method.

### Means for Solving the Problems

The present inventors extensively investigated in view of the problems described above, and found that decidua-derived mesenchymal cells, which can be obtained easily in large amounts as a byproduct of delivery, and extracellular matrixes obtained from decidua-derived mesenchymal cells, have potent cell maintenance culture supporting activity. The inventors conducted further investigations based on this finding, and have established a technique for performing maintenance culture of human pluripotent stem cells safely and efficiently using the cells or the matrixes, thus developing the present invention. Accordingly, the present invention is as follows:

(1) A method of culturing pluripotent stem cells in the presence of decidua-derived cells or an extracellular matrix derived from the cell.
(2) The method according to (1) above, wherein the decidua-derived cell is a mesenchymal cell.
(3) The method according to (1) above, wherein both the decidua-derived cell and the pluripotent stem cells are derived from a mammal of the same species.
(4) The method according to (3) above, wherein the mammal is a human.
(5) The method according to (1) above, wherein the pluripotent stem cells are human ES cells or human iPS cells.
(6) A culture agent for pluripotent stem cells, containing decidua-derived cells or an extracellular matrix derived from the cell.
(7) The agent according to (6) above, wherein the decidua-derived cell is a mesenchymal cell.
(8) The agent according to (6) above, wherein both the decidua-derived cell and the pluripotent stem cells are derived from a mammal of the same species.
(9) The agent according to (8) above, wherein the mammal is a human.
(10) The agent according to (6) above, wherein the pluripotent stem cells are human ES cells or human iPS cells.
(11) A container for culturing pluripotent stem cells, coated with an extracellular matrix from decidua-derived cells.
(12) The container according to (11) above, wherein the decidua-derived cell is a mesenchymal cell.
(13) The container according to (11) above, wherein both the decidua-derived cell and the pluripotent stem cells are derived from a mammal of the same species.
(14) The container according to (13) above, wherein the mammal is a human.
(15) The container according to (11) above, wherein the pluripotent stem cells are human ES cells or human iPS cells.
(16) A kit for culturing pluripotent stem cells, comprising (i) and (ii) below:
(i) a culture agent for pluripotent stem cells, comprising decidua-derived cells or an extracellular matrix derived from the cell; and
(ii) an explanatory document stating that the kit should be used, or can be used, for culturing pluripotent stem cells.
(17) A kit for culturing pluripotent stem cells, comprising (i) and (ii) below:
(i) a container for culturing pluripotent stem cells, coated with an extracellular matrix from decidua-derived cells; and
(ii) an explanatory document stating that the kit should be used, or can be used, for culturing pluripotent stem cells.

### EFFECT OF THE INVENTION

According to the present invention, maintenance culture of human pluripotent stem cells can be performed safely and efficiently. The culture agent and container for pluripotent stem cells of the present invention afford sufficient quantitative supplies because the decidua, which is the source material, can be obtained in large amounts relatively easily at the time of delivery. Furthermore, uniform quality control (activity and safety) can be achieved since the culture agent and the container of the present invention used for pluripotent stem calls can be prepared in large amounts at one time. Additionally, containers (e.g., culture dish) coated with an extracellular matrix from decidua-derived cells, used in the present invention, can be stored for a long time (e.g., 8 months or more) in a refrigerator; in preparation for clinical application, the containers may be previously subjected to adequate testing on safety and activity to ensure controlled quality. This quality control is also important in cases where the containers are used for drug discovery, toxicity testing and the like.

### BRIEF DESCRIPTION OF THE DRAWINGS

FIG. 1 is a schematic representation of the procedures for preparing decidua-derived mesenchymal cells.

FIG. 2 is a phase-contrast photomicrograph of a decidua-derived mesenchymal cell.

FIG. 3 is a graphic representation of proliferation curves for cells of human fetal membrane origin. DMC: human decidua-derived mesenchymal cells, AEC: human amniotic epithelial cells, AMC: human amniotic mesenchymal cells.

FIG. 4 is a schematic representation of the procedures for preparing an extracellular matrix from decidua-derived mesenchymal cells.

### EMBODIMENTS FOR CARRYING OUT THE INVENTION

All technical terms and scientific terms used herein have the same meanings as those generally understood by those skilled in the technical field to which the present invention pertains unless otherwise specified. Optionally chosen methods and materials similar or equivalent to those described herein can be used in carrying out or testifying the present invention. Preferable methods and materials are described below. The disclosures in all publications and patents mentioned herein are incorporated herein by reference for the purpose of, for example, describing and disclosing the products and methodologies described in publications that can be used in relation to the inventions described.

The present invention provides a method of culturing pluripotent stem cells in the presence of decidua-derived cells or an extracellular matrix derived from the cell.

The “decidua-derived cell” used in the present invention may be any cell derived from the decidua; examples include primary culture cells isolated from the decidua, established cell lines thereof and the like. A cell line can be established using a method in common use in the art. Specifically, an established cell line can be derived by cell cloning by limited dilution or by artificially conferring a proliferating capability by gene transfer and the like.

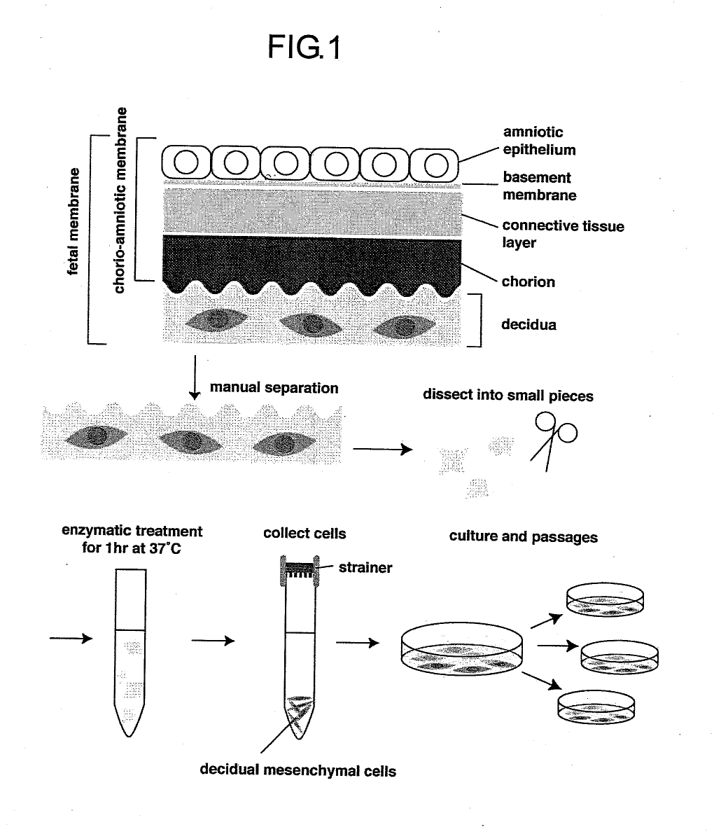

The human fetal membrane is configured mainly with three layers: the amnion (epithelial tissue and mesenchymal system; inner layer), the chorion (middle layer), and the decidua (outer layer) (see FIG. 1). The amnion and chorion are of fetal origin, whereas the decidua is of maternal origin. Examples of cells derived from the fetal membrane include amniotic epithelial cells (AEC), amniotic (including some chorion) mesenchymal cells (AMC), decidual mesenchymal cells (DMC) and the like. In the present invention, decidua-derived cells are used since they can be collected and prepared in large amounts and are highly capable of proliferation. As stated below, decidual mesenchymal cells, which have higher maintenance culture supporting activity for pluripotent stem cells, are particularly preferable.

Here, “maintenance culture” of cells means culturing pluripotent stem cells in a way that allows their proliferation and passage in an undifferentiated state.

A decidua can be prepared by a method known per se. Usually, decidual tissue of placental accessory tissue is obtained during delivery, dissected with scissors and the like, and washed with phosphate-buffered saline (PBS) or physiological saline to remove blood components and unwanted tissue, after which it is divided into pieces of appropriate size and stored in an appropriate buffer solution. The subject of collection of the decidua is preferably negative for infectious diseases (e.g., hepatitis B, hepatitis C, syphilis, human immunodeficiency virus). The presence or absence of an infectious disease can be determined by a method known per se, for example, serological testing.

Decidua-derived cells can be recovered from decidual tissue mechanically and/or enzymatically. For example, when decidua-derived mesenchymal cells are obtained from the decidua, decidual tissue is treated with an enzyme (e.g., collagenase, dispase, trypsin), and stirred at 20 to 40° C., preferably about 37° C., a temperature close to normal body temperature, for 15 to 120 minutes, preferably about 60 minutes, whereby decidua-derived mesenchymal cells can be isolated and recovered.

It can be determined whether the cells obtained are desired decidua-derived cells by checking for the presence or absence of a phenotype specific for the cells. For example, decidua-derived mesenchymal cells can be identified by the mode of expression of actin in the cells or the expression of vimentin. The mode of expression of actin can be determined by fluorescent staining with phalloidin, and the expression of vimentin can be confirmed by immunostaining with anti-vimentin antibody. Furthermore, it is preferable that non-identity as AMC and the absence of AMC contamination be continued by confirming negativity for HLA-G, which is a marker for amniotic tissue. Confirmation of non-identity as epithelial cells can be achieved by confirming negativity for CK19, which is a marker for epithelial cells. Vimentin is an intermediate filament characteristic of mesenchymal cells. When mesenchymal cells are fluorescently stained with phalloidin, a stress fiber structure is observed.

Decidua-derived cells useful in the present invention are cells derived from an optionally chosen warm-blooded animal, preferably a mammal. Examples of the mammal include mice, rats, guinea pigs, hamsters, rabbits, cats, dogs, sheep, pigs, bovines, horses, goat, monkeys, humans, and the like, with preference given to humans. In particular, cells derived from a mammal of the same species as the below-mentioned pluripotent stem cells used in the culturing method of the present invention, preferably from a human, can be used.

In the present invention, to culture pluripotent stem cells “in the presence of decidua-derived cells or an extracellular matrix derived from the cell” refers to culturing pluripotent stem cells in a medium containing at least decidua-derived cells or an extracellular matrix derived from the cell. Here, “to culture pluripotent stem cells” means that the pluripotent stem cells are allowed to propagate while retaining the pluripotency thereof, i.e., in an undifferentiated state. Hence, this is defined as being identical to “maintenance culture of pluripotent stem cells”. The fact that pluripotent stem cells are in an undifferentiated state can be confirmed by determining whether or not the maintenance-cultured cells retain their original pluripotency by examining the expression of the undifferentiation markers Oct3/4, SSEA4, TRA-1-60, Nanog, alkaline phosphatase and the like. The expression of each undifferentiation marker can be checked by a method in common use in the art, or a method based thereon. When the expression of each marker is confirmed at the protein level, a specific antibody against each marker protein can be used. When the expression is confirmed at the gene level, a specific probe or primer for each marker gene can be used. Specific antibodies and specific probes/primers are commercially available, or can be prepared on the basis of known amino acid sequences or base sequences according to a conventional method.

The medium used in the culturing method of the present invention can be prepared using a medium used for culturing an animal cell as a basal medium. Any basal medium available for culturing an animal cell can be used; examples include BME medium, BGJb medium, CMRL 1066 medium, Glasgow MEM medium, Improved MEM Zinc Option medium, IMDM medium, Medium 199 medium, Eagle's MEM medium, αMEM medium, DMEM medium, Ham's medium, RPMI 1640 medium, Fischer's medium, a mixed medium thereof and the like.

Although the medium used in the culturing method of the present invention can be a serum-containing medium or a serum-free medium, a serum-free medium is preferable from the viewpoint of assuring the safety of cell transplantation by eliminating heterologous components. Here, a serum-free medium means a medium not containing an unadjusted or unpurified serum; a medium contaminated with a purified blood-derived component or animal tissue-derived component (e.g., growth factor) is deemed a serum-free medium. Examples of such serum-free media include a serum-free medium supplemented with an appropriate amount (e.g., 1-20%) of commercial product KNOCKOU™ SR, a serum-free medium supplemented with insulin and transferrin [e.g., CHO-S-SFM II (manufactured by GIBCO BRL), Hybridoma-SFM (manufactured by GIBCO BRL), eRDF Dry Powdered Media (manufactured by GIBCO BRL), UltraCULTURE™ (manufactured by BioWhittaker), UltraDOMA™ (manufactured by BioWhittaker), UltraCHO™ (manufactured by BioWhittaker), UltraMDCK™ (manufactured by BioWhittaker), ITPSG medium (Cytotechnology, 5, S17 (1991)), ITSFn medium (Proc. Natl. Acad. Sci. USA, 77, 457 (1980)), mN3 medium (Mech. Dev., 59, 89 (1996) and the like), a medium supplemented with a cell-derived factor (e.g., a medium supplemented with a culture supernatant of pluripotent teratocarcinoma cell PSA1 [Proc. Natl. Acad. Sci. USA, 78, 7634 (1981)] and the like. STEMPRO hESC SFM (manufactured by Invitrogen), which has been developed for proliferation of human ES cells, is also preferably used.

The medium for the present invention may also contain a serum substitute or not. The serum substitute can be, for example, one containing as appropriate albumins (e.g., lipid-rich albumins), transferrin, fatty acids, insulin, collagen precursor, trace elements, 2-mercaptoethanol, 3′-thiolglycerol, or equivalents thereof and the like. This serum substitute can be prepared by, for example, a method described in WO98/30679. Also, to carry out the method of the present invention more conveniently, a commercially available serum substitute can be utilized. Examples of such commercially available serum substitutes include Knockout Serum Replacement (KSR), Chemically-defined Lipid concentrated (manufactured by Gibco), and Glutamax (manufactured by Gibco).

The medium of the present invention can also contain fatty acids or lipids, amino acids (e.g., non-essential amino acids), vitamins, growth factors, cytokines, antioxidants, 2-mercaptoethanol, pyruvic acid, buffering agents, inorganic salts and the like. For example, 2-mercaptoethanol can be used at any concentrations suitable for the cultivation of stem cells, e.g., at about 0.05 to 1.0 mM, preferably about 0.1 to 0.5 mM.

Any container for cell culture can be used to culture pluripotent stem cells; examples include flasks, tissue culture flasks, dishes, Petri dishes, tissue culture dishes, multi-dishes, microplates, micro-well plates, multi-plates, multi-well plates, chamber slides, schale, tubes, trays, culturing bags, and roller bottles.

In an embodiment of the culturing method of the present invention, pluripotent stem cells are cultured in the presence of decidua-derived cells as described above; preferably, the decidua-derived cells are used as support cells (feeder cells) for the pluripotent stem cells. Specifically, the pluripotent stem cells are seeded onto the decidua-derived cells and cultured. Seeding density and culture conditions for the decidua-derived cells as the feeder cells, and seeding density and culture conditions for the pluripotent stem cells, and the like are set as appropriate according to the choice of pluripotent stem cells to be cultured and the like; for example, the same as with the use of MEF as the feeder cell applies.

More specifically, for culturing pluripotent stem cells in the presence of decidua-derived cells, a method can be used in which the pluripotent stem cells are suspended in an appropriate medium (described above), seeded onto a separately prepared feeder layer of decidua-derived cells at a cell density of 3000 to 16000 cells/cm², and cultured at 20 to 40° C., preferably 3.7° C., in a CO₂ incubator being aerated with several percents, preferably 5%, of carbon dioxide for 2 to 7 days. The feeder layer of decidua-derived cells used is normally one obtained by seeding decidua-derived cells at a cell density of 4000 to 40000 cells/cm², and culturing the cells at 20 to 40° C., preferably 37° C., in a CO₂ incubator being aerated with several percents, preferably 5%, of carbon dioxide for 1 to 7 days until they become confluent. It is desirable that the feeder layer be previously deactivated by a treatment with mitomycin C.

In an embodiment of the culturing method of the present invention, pluripotent stem cells are cultured in the presence of an extracellular matrix from decidua-derived cells. Preferably the pluripotent stem cells are cultured on an extracellular matrix derived from decidua-derived cells.

The “extracellular matrix” derived from a decidua-derived cell, used in the present invention, is an extracellular matrix obtained from a decidua-derived cell, particularly a decidua-derived mesenchymal cell.

An extracellular matrix can be prepared from decidua-derived cells (described above) as the source material by a method known per se. The matrix can usually be obtained by removing cell components by means of an EDTA solution, a surfactant (deoxycholic acid, sodium dodecyl sulfate, polyoxyethylene sorbitan fatty acid ester and the like) or the like, and recovering the components remaining on a container. Briefly, by removing the cell components from the decidua-derived cells cultured on the container as described above to leave the extracellular matrix components on the container, recovery of the extracellular matrix and coating of the surface of the container with the matrix can be achieved simultaneously. This operation is preferable in that the matrix can be recovered without a loss and without denaturation.

More specifically, for culturing pluripotent stem cells on an extracellular matrix derived from decidua-derived cells, a method can be used in which the pluripotent stem cells are suspended in an appropriate medium (described above), seeded onto a culture container, which is separately prepared and coated with an extracellular matrix from decidua-derived cells, at a cell density of 3000 to 16000 cells/cm², and cultured at 20 to 40° C., preferably 37° C., in a CO₂ incubator being aerated with several percents, preferably 5%, of carbon dioxide for 2 to 7 days.

In the present invention, “a pluripotent stem cell” refers to a cell that can be cultured in vitro, and that possesses multipotency for differentiation into all types of cells that constitute a living organism. Specifically, such cells include embryonic stem cells (ES cells), pluripotent stem cells of embryonic primordial germ cell origin (EG cells: Proc Natl Acad Sci USA. 1998, 95:13726-31), pluripotent testis-derived germline stem cells of testis origin (GS cells: Nature. 2008, 456:344-9), induced pluripotent stem cells (iPS cells) of somatic cell origin and the like.

ES cells derived from an optionally chosen warm-blooded animal, preferably a mammal, can be used. Examples of the mammal include mice, rats, guinea pigs, hamsters, rabbits, cats, dogs, sheep, pigs, bovines, horses, goat, monkeys, humans, and the like, with preference given to humans. In particular, ES cells derived from a mammal of the same species as the aforementioned decidua-derived cell used in the culturing method of the present invention, preferably from a human, can be used.

Specifically, examples of ES cells useful in the method of the present invention include ES cells of a mammal or the like established by culturing a pre-implantation early embryo (hereinafter, abbreviated to “ES cells I”), ES cells established by culturing an early embryo prepared by nuclear-transplanting the nucleus of a somatic cell (hereinafter, abbreviated to “ES cells II”), and ES cells prepared by modifying a gene on the chromosome of ES cells I or II using a gene engineering technique (hereinafter, abbreviated to “ES cells III”).

More specifically, as ES cells I, ES cells established from an inner cell mass that constitutes an early embryo, cells isolated from a cell population possessing pluripotency present in the differentiation stage of an early embryo before or just after implantation (for example, primordial ectoderm) (EpiStem cells: Nature. 2007 448:191-5, Nature. 2007 448:196-9), or cells obtained by culturing these cells and the like can be mentioned.

ES cells I can be prepared by culturing a pre-implantation early embryo according to a method described in the literature [Manipulating the Mouse Embryo A Laboratory Manual, Second Edition, Cold Spring Harbor Laboratory Press (1994)].

ES cells II can be prepared, for example, as described below, using, for example, methods reported by Wilmut et al. [Nature, 385, 810 (1997)], Cibelli et al. [Science, 280, 1256 (1998)], Akira Iritani et al. [Protein, Nucleic Acid and Enzyme, 44, 892 (1999)], Baguisi et al. [Nature Biotechnology, 17, 456 (1999)], Wakayama et al. [Nature, 394, 369 (1998); Nature Genetics, 22, 127 (1999); Proc. Natl. Acad. Sci. USA, 96, 14984 (1999)], Rideout III et al. [Nature Genetics, 24, 109 (2000)] and others.

By reprogramming the nucleus of a mammalian cell after being extracted from the cell (an operation to restore the nucleus to a state to resume development), initiating development using a method wherein the nucleus is injected into an enucleated mammalian unfertilized egg, and culturing the egg that has started development, an egg that has the nucleus of another somatic cell, and has begun normal development, is obtained.

A plurality of methods are known to enable reprogramming of the nucleus of a somatic cell. For example, the nucleus can be reprogrammed by inducing the cell cycle to enter a resting phase state (phase G0 or phase G1) by culturing the nucleus donor cell for 3 to 10 days, preferably 5 days after replacing the medium from a medium containing 5 to 30%, preferably 10%, of fetal calf serum (e.g., M2 medium) with an oligotrophic medium containing 0 to 1%, preferably 0.5%, of fetal calf serum.

The nucleus can also be reprogrammed by injecting the nucleus of the nucleus donor cell into an enucleated unfertilized egg of a mammal of the same species, and culturing the egg for several hours, preferably about 1 to 6 hours.

The reprogrammed nucleus is able to begin to develop in the enucleated unfertilized egg. A plurality of methods are known to initiate the development of the reprogrammed nucleus in the enucleated unfertilized egg. By transplanting a nucleus reprogrammed by inducing the cell cycle to enter a resting phase state (phase G0 or phase G1) into an enucleated unfertilized egg of a mammal of the same species by the electrofusion method and the like, the egg can be activated and allowed to begin to develop.

A nucleus reprogrammed by injecting the nucleus into an enucleated unfertilized egg of a mammal of the same species is again transplanted to an enucleated unfertilized egg of a mammal of the same species by a method using a micromanipulator or the like, and stimulated with an egg activator (e.g., strontium and the like), and thereafter treated with an inhibitor of cell division (e.g., cytochalasin B and the like) to suppress the release of the second polar body, whereby development can be initiated. This method is suitable when the mammal is, for example, a mouse or the like.

Provided that an egg that once began to develop is obtained, ES cells can be acquired using publicly known methods described in Manipulating the Mouse Embryo A Laboratory Manual, Second Edition, Cold Spring Harbor Laboratory Press (1994); Gene Targeting, A Practical Approach, IRL Press at Oxford University Press (1993); Biomanual Series 8 Gene Targeting, Preparation of Mutant Mice Using ES Cells, Yodosha (1995) and the like.

ES cells III can be prepared by, for example, homologous recombination technology. Examples of the gene on the chromosome to be modified in preparing ES cells III include histocompatibility antigen genes, genes related to diseases based on nervous system cell disorders and the like. A modification of the target gene on the chromosome can be performed using methods described in Manipulating the Mouse Embryo A Laboratory Manual, Second Edition, Cold Spring Harbor Laboratory Press (1994); Gene Targeting, A Practical Approach, IRL Press at Oxford University Press (1993); Biomanual Series 8 Gene Targeting, Preparation of Mutant Mice Using ES Cells, Yodosha (1995) and the like.

Specifically, for example, the genomic gene of a target gene to be modified (e.g., histocompatibility antigen genes, disease-related genes and the like) is isolated, and a target vector for homologous recombination of the target gene is prepared using the genomic gene isolated. The target vector prepared is introduced into ES cells, and cells undergoing homologous recombination between the target gene and the target vector are selected, whereby ES cells having a modified gene on the chromosome can be prepared.

The genomic gene of a target gene can be isolated by publicly known methods described in Molecular Cloning, A Laboratory Manual, Second Edition, Cold Spring Harbor Laboratory Press (1989), Current Protocols in Molecular Biology, John Wiley & Sons (1987-1997) and elsewhere. The genomic gene of a target gene can also be isolated using a genomic DNA library screening system (manufactured by Genome Systems), Universal GenomeWalker™ Kits (manufactured by CLONTECH) and the like.

Preparation of a Target Vector for Homologous recombination of a target gene and efficient selection of a homologous recombinant can be achieved according to methods described in Gene Targeting, A Practical Approach, IRL Press at Oxford University Press (1993); Biomanual Series 8 Gene Targeting, Preparation of Mutant Mice Using ES Cells, Yodosha (1995) and elsewhere. The target vector used may be any one of the replacement type and the insertion type; regarding methods of selection, positive selection, promoter selection, negative selection, poly A selection and the like can be used.

A desired homologous recombinant can be selected from among sorted cell lines, Southern hybridization, PCR and the like for genomic DNA can be mentioned.

ES cells are available from specified organizations, and commercial products may be purchased. For example, the human ES cells KhES-1, KhES-2 and KhES-3 are available from the Institute for Frontier Medical Sciences, Kyoto University.

iPS cells derived from an optionally chosen warm-blooded animal, preferably a mammal, can be used. Examples of the mammal include mice, rats, guinea pigs, hamsters, rabbits, cats, dogs, sheep, pigs, bovines, horses, goat, monkeys, humans, and the like. In particular, cells derived from a mammal of the same species as the aforementioned decidua-derived cell used in the culturing method of the present invention, preferably from a human, can be used.

Specifically, examples of iPS cells used in the method of the present invention include cells that have acquired multipotency like that of ES cells, obtained by transferring a plurality of genes into somatic cells such as skin cells; examples include iPS cells obtained by introducing the Oct3/4 gene, Klf4 gene, C-Myc gene and Sox2 gene, iPS cells obtained by introducing the Oct3/4 gene, Klf4 gene and Sox2 gene (Nat Biotechnol 2008; 26: 101-106) and the like.

iPS cells are available from specified organizations (RIKEN BioResource Center, Kyoto University).

The present invention provides a culture agent for pluripotent stem cells, comprising decidua-derived cells or an extracellular matrix derived from the cell.

The decidua-derived cell or extracellular matrix derived from the cell that can be contained in the culture agent of the present invention may be the same as the above-described decidua-derived cell or extracellular matrix derived from the cell used in the method of the present invention for culturing pluripotent stem cells. The culture agent of the present invention may contain ingredients required for culturing pluripotent stem cells, in addition to the decidua-derived cell or an extracellular matrix derived from the cell. For example, in addition to decidua-derived cells and an extracellular matrix derived from the cell, a medium and other ingredients (amino acids, pyruvic acid, 2-mercaptoethanol, cytokines, growth factors and the like), which are required for culturing pluripotent stem cells, may be contained in the culture agent. Here, the medium contained in the culture agent may be as described above.

The present invention provides a container for culturing pluripotent stem cells, coated with an extracellular matrix from decidua-derived cells.

The container used here is as described above; examples include flasks, tissue culture flasks, dishes, Petri dishes, tissue culture dishes, multi-dishes, microplates, micro-well plates, multi-plates, multi-well plates, chamber slides, schale, tubes, trays, culturing bags, and roller bottles. Preference is given to dishes, Petri dishes, tissue culture dishes, multi-dishes, microplates, micro-well plates, multi-plates, multi-well plates, and the like. The extracellular matrix from decidua-derived cells used to coat the container may be as described above. Coating of the container with an extracellular matrix can normally be performed according to a method in use in the art; for example, the coating can be achieved by treating the decidua-derived cell cultured on a culture dish with EDTA solution and the like to remove the cell components, to leave the extracellular matrix components on the culture dish. The thus-obtained container coated with the extracellular matrix can be stored in a refrigerator for 8 months or more.

The present invention further provides a kit for culturing pluripotent stem cells. A kit of the present invention can comprise the above-described pluripotent stem cell culture agent or container for culturing pluripotent stem cells, and other components separately (i.e., in non-mixed mode). For example, a kit of the present invention can be provided in a form wherein the individual components are housed in separate holders. Examples of other components that can be contained in a kit of the present invention include a substance for identification or measurement (detection or quantitation) of pluripotent stem cells (e.g., antibody against cell marker), medium, plasmid for gene recombination and a drug for its selection. The kit may contain an explanatory document stating that the kit should be used, or can be used, for culturing pluripotent stem cells.

The culturing method, culture agent, culture container and kit of the present invention can also be suitably used for dissociation culture of pluripotent stem cells. Dissociation culture refers to culturing cells that have been undergone cell dissociation treatment (dissociated cells); examples of dissociated cells include single cells and cells in the form of a small cell mass consisting of several (e.g., about 2 to 20) cells. Dissociation of pluripotent stem cells can be achieved by a method known per se. Examples of such methods include treatments with a chelating agent (e.g., EDTA), enzyme (e.g., trypsin, collagenase) and the like, and operations such as mechanical detachment (e.g., pipetting).

### EXAMPLES

The present invention is hereinafter described in more detail with reference to Examples, which, however, do not limit the scope of the present invention by any means. The reagents, apparatuses and materials used in the present invention are commercially available unless otherwise specified.

### Example 1

### Maintenance Culture of Human ES Cells on Mesenchymal cells of maternal origin from human fetal membrane (decidua)

### 1. Preparation of Decidua-Derived Mesenchymal Cells (DMC)

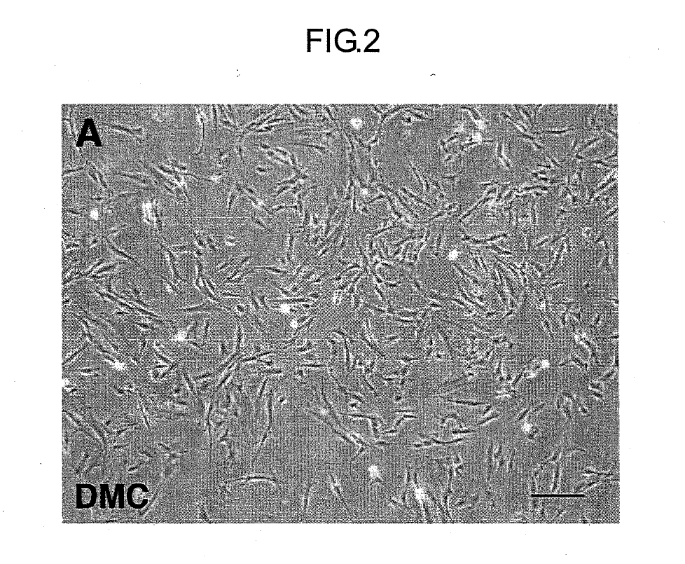

Complying with informed consent previously obtained from a mother, the human fetal membrane of placenta accessory tissue during delivery was obtained, and the tissue of the decidual portion was manually recovered therefrom. The human decidual tissue was dissected with scissors, after which it was warmed at 37° C. in a solution comprising PBS supplemented with 0.1% collagenase I solution, 0.01% DNase I, and 0.1% dispase (all manufactured by Invitrogen/Gibco-BRL), using a constant-temperature chamber equipped with a shaker for 60 minutes, and the cells were separated and cultured. The cells recovered were identified as mesenchymal cells by examination of their morphology (FIG. 2), and by actin staining with phalloidin and vimentin immunostaining with anti-vimentin antibody. Negativity for HLA-G, which is a marker for amniotic tissue and fetal tissue, was also confirmed by immunostaining, whereby its identity as a decidua-derived component. Furthermore, negativity for epithelial cell marker CK19 was confined by immunostaining with anti-CK19 antibody. Cultivation was performed using a D-MEM-F12 medium (Sigma D8437) supplemented with 10% fetal bovine serum (FBS) on a plastic culture dish at 37° C. in the presence of 5% CO₂. While the medium was replaced with a fresh supply twice a week, the cells were subjected to passage culture using trypsin-EDTA (0.05%, Invitrogen).

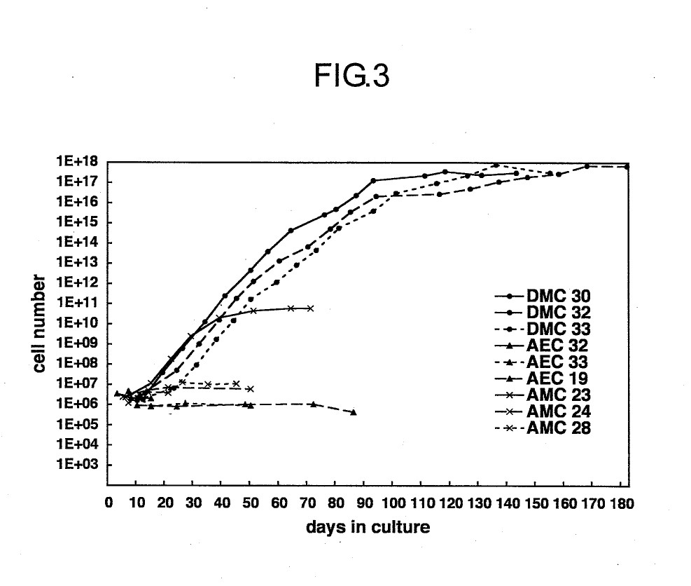

Separately, amniotic epithelial cells (AEC) and amniotic mesenchymal, cells (AMC) were prepared from the fetal membrane. The AEC, AMC, and DMC were examined for cell proliferation capability. Three lines of each cell type were used. These cells were seeded to a culture dish at a density of 2×10⁵ cells/100 mm for each passage, and cultured until they became confluent. After each passage, the cells were re-suspended, counted, and re-seeded to a new culture dish at the same cell density. Total cell counts were calculated from the cell counts obtained (FIG. 3). As a result, DMC was shown to have higher proliferating capacity than AEC and AMC. This property is advantageous in that a larger number of cells are secured.

### 2. Preparation of Human ES Cells

The human ES cells used in the experiments (KhES1, KhES3) were embryonic stem cells of human blastocyst origin established at Norio Nakatsuji's laboratory in the Institute for Frontier Medical Sciences, Kyoto University, which were kindly supplied in compliance with the Guidelines for Derivation and Utilization of Human ES Cells. According to the method of Nakatsuji's laboratory (Biochem Biophys Res Commun 2006; 345:926-932), undifferentiated human ES cells were subjected to maintenance culture on a plastic culture dish with mouse embryonic fibroblasts (deactivated by mitomycin treatment; MEF) as feeder cells seeded thereon. Specifically, the culture broth used was prepared by adding to D-MEM-F12 (Sigma D6421) a final concentration of 20% of KSR (Invitrogen/Gibco-BRL), 0.1 mM NEAA (non-essential amino acids; Invitrogen/Gibco-BRL), 2 mM L-glutamine, 5 ng/ml human basic FGF (Wako) and 0.1 mM 2-mercaptoethanol; the ES cells were cultured at 37° C. in the presence of 2% CO₂.

Passage was performed every 3 to 4 days; human ES cell colonies were dissociated from the feeder layer using a dissociating liquid (PBS supplemented with 0.25% trypsin, 0.1 mg/ml collagenase IV solution, 1 mM CaCl₂, and a final concentration of 20% of KSR; all manufactured by Invitrogen/Gibco-BRL), and divided into about 20 small masses by pipetting, after which they were seeded onto a new feeder layer. Until use in the experiments, the human ES cells was cultured using MEF as feeder cells as in a conventional method (Science 1998; 282:1145-1147, Nat Biotechnol 2000; 18:399-404, Cell 2007; 131:861-872).

### 3. Maintenance Culture of Human ES Cells on DMC

In an experiment for analyzing the feeder activity of DMC, DMCs were seeded at a density of 400,000 cells/6 cm culture dish, and treated with mitomycin C (10 μg/ml), after which they were used as the feeder cells for culturing human ES cells. The other culturing operations were performed in the same manner as the above-described conventional maintenance culture using MEF as the feeder.

The human ES cells proliferated well on the DMC feeder, and increased their cell count 2,800 folds in 40 days. The ES cell colonies formed on the DMC feeder were found to express the undifferentiation markers Oct3/4, SSEA4, TRA-1-60, Nanog and the like, nearly uniformly, demonstrating that maintenance culture of undifferentiated ES cells was efficiently performed.

The expression of each undifferentiation marker was confirmed using a specific antibody against the marker. All antibodies used are commercially available, and were used as directed in the manufacturers' instruction manuals.

### Example 2

### Maintenance Culture of Human ES Cells on Extracellular Matrix Extracted from Human Decidua-Derived Mesenchymal Cells

### (Methods)

Human decidua-derived mesenchymal cells were prepared as described in Example 1. The human decidua-derived mesenchymal cells were seeded to a plastic culture dish coated with 0.1% gelatin at a density of 3.5×10⁴ cells/cm², and cultured for 3 days while a confluent state was maintained. After washing with PBS, the cultured cells were treated with deoxycholic acid (treatment with 0.5% sodium deoxycholate/10 mM Tris-HCl, pH 8.0, added to the culture dish, at 4° C. for 30 minutes) to lyse the cell components. The extracellular matrix components remaining on the culture dish were washed with PBS. For culturing the human decidua-derived mesenchymal cells, a D-MEM-F12 medium supplemented with 10% fetal bovine serum, or a D-MEM-F12 medium supplemented with N2 supplement (Invitrogen)+20 ng/ml human recombinant FGF-2 (Peprotech)+20 ng/ml human recombinant EGF (Peprotech)+5 μg/ml heparin (maintenance culture broth), was used. Hereinafter, for the sake of convenience, the former maintenance culture broth containing serum is referred to as the serum-containing maintenance culture broth, and the serum-free maintenance culture broth as the maintenance culture broth free of serum.

As directed in Example 1, human ES cell masses were sown to this culture dish on which the extracellular matrix remained, and maintenance culture was performed. The culture broth used in this step was a culture supernatant prepared by acclimating the above-described maintenance culture broth with mouse embryonic fibroblasts (MEF) by a conventional method (Nat Biotechnol 2001; 19:971-974).

For control, culture dishes coated with gelatin (Siyma, 0.1%), fibronectin (Invitrogen, 5 μg/cm²) or Matrigel (BD, 1:30) by incubation at room temperature for 1 hour were used.

Culture dishes on which the extracellular matrix remained, i.e., culture dishes coated with the extracellular matrix, were stored in a semi-dry condition at 4° C. until use.

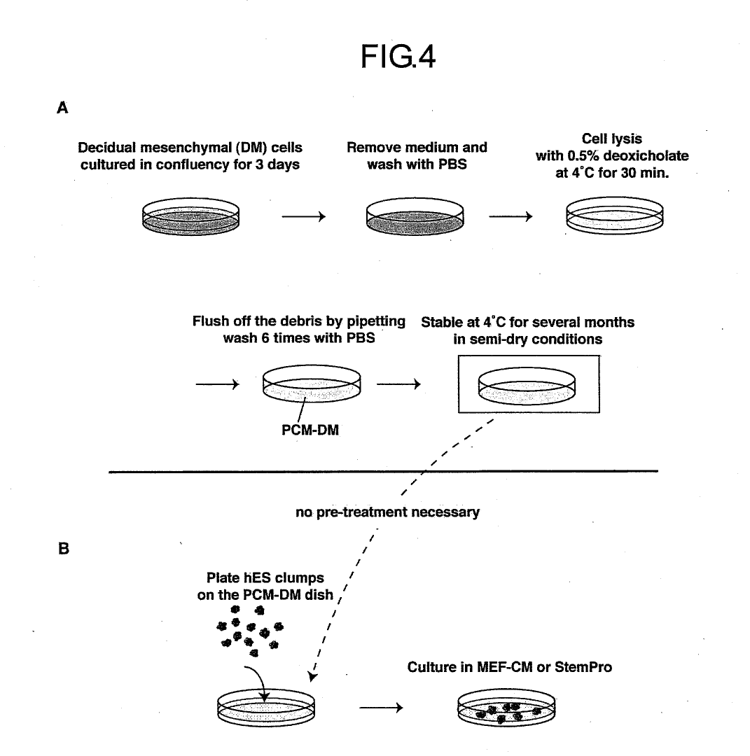

The procedures for preparing the extracellular matrix are schematically shown in FIG. 4.

### (Results)

The human ES cells proliferated well on the extracellular matrix from human decidua-derived mesenchymal cells, and increased their cell count 15,000,000 folds compared to the starting level 59 days later. Meanwhile, on Matrigel, the cell count increased 1,500,000 folds compared to the starting level 59 days later. Meanwhile, almost no cell proliferation was observed on gelatin. Although a little cell proliferation was observed on fibronectin, the extent was considerably lower than that of cell proliferation on the extracellular matrix from decidua-derived mesenchymal cells.

Whether the medium used was the serum-containing maintenance culture broth or the serum-free maintenance culture broth, the extracellular matrix prepared exhibited potent maintenance culture supporting activity for human ES cells.

The human ES cells cultured on the extracellular matrix from human decidua-derived mesenchymal cells exhibited a high nucleus/cytoplasm ratio, had a small size, and formed a flat colony of high cell density. Furthermore, the cells were strongly positive for the undifferentiation markers Oct3/4, SSEA4, TRA-1-60, Nanog, alkaline phosphatase and the like.

These findings show that the extracellular matrix from human decidua-derived mesenchymal cells exhibits a maintenance culture supporting activity for human ES cells, equivalent to, or higher than, that of Matrigel. Even when a culture supernatant of human decidua-derived mesenchymal cells was used in place of the culture supernatant of MEF, the extracellular matrix from human decidua-derived mesenchymal cells exhibited a maintenance culture support activity equivalent to, or higher than, that of Matrigel.

### Example 3

### Maintenance Culture of Human ES Cells on Extracellular Matrix from Human Decidua-Derived Mesenchymal Cells Using Chemically Synthetic Medium

### (Methods)

Preparation of an extracellular matrix from human decidua-derived mesenchymal cells and maintenance culture of human ES cells were performed in the same manner as Example 2 except for the culture broth. In place of the MEF culture supernatant, the STEMPRO hESC SFM culture broth (Invitrogen Co.), a chemically synthetic medium, was used as the culture broth.

Further more, maintenance culture of human ES cells was performed in the same manner using culture dishes coated with an extracellular matrix from human decidua-derived mesenchymal cells, stored in a refrigerator (4° C.) for 3 weeks and 8 months.

### (Results)

In the culture on the extracellular matrix from human decidua-derived mesenchymal cells using the STEMPRO hESC SFM culture broth, human ES cells proliferated well, and the cell count increased 34,000,000 folds compared to the starting level 44 days later. The human ES cells thus cultured were strongly positive for the undifferentiation markers Oct3/4, SSEA4, TRA-1-60, Nanog, alkaline phosphatase and the like.

These results demonstrate that the extracellular matrix from human decidua-derived mesenchymal cells exhibits an excellent maintenance culture supporting activity for pluripotent stem cells even when a chemically synthetic medium is used.

Regarding the maintenance culture supporting activity for pluripotent stem cells, a comparison was made between the use of a culture dish stored in a refrigerator (4° C.) for 3 weeks and the use of a culture dish stored for 8 months; no difference was found. This result demonstrates that the extracellular matrix from decidua-derived mesenchymal cells of the present invention retains its maintenance culture supporting activity even after storage in a refrigerator for at least 8 months.

### Example 4

### Confirmation of Pluripotency of Human ES Cells after Passage Maintenance Culture on Extracellular Matrix from Human Decidua-Derived Mesenchymal Cells (Methods)

Maintenance culture was performed on human ES cells for passages as described in Example 3, after which their pluripotency was checked by immunostaining, in vitro differentiation induction, and the teratoma formation method.

The reagents, such as antibodies, used in the immunostaining are all commercially available, and were used as directed in the manufacturers' instruction manuals.

For the in vitro differentiation induction, the serum treatment method by adhesion culture (Nat Biotechnol 2007; 25:681-686) or the SDIA method by adhesion culture on PA6 cells (Neuron 2000; 28:31-40) was used.

In the teratoma formation method, 500,000 human ES cells were transplanted to SCID mouse testis, and tumorigenesis was examined.

### (Results)

Even after for 10 passages, the human ES cells were strongly positive for the undifferentiation markers Oct3/4, SSEA4, TRA-1-60, Nanog, alkaline phosphatase and the like. In the in vitro differentiation induction, the adhesion culture confirmed differentiation into cells positive for HNF3s/E-cadherin, which are epithelial cells of endoderm origin, and cells positive for Brachyury, which are cells of mesoderm origin, and the SDIA method confirmed differentiation into nerve progenitors positive for Nestin/Pax6. Twelve weeks after transplantation to the testis, development of teratomas, including brain tissue, cartilage tissue, secretory mucosal tissue and the like, was confirmed. These results showed that the human ES cells maintenance-cultured on the extracellular matrix from human decidua-derived mesenchymal cells retained the pluripotency even after the repeated passages.

### Example 5

### Maintenance Culture of Single Dissociated Human ES Cells on Extracellular Matrix from Human Decidua-Derived Mesenchymal Cells

### (Methods)

Single dissociated human ES cells were cultured using an extracellular matrix from human decidua-derived mesenchymal cells and STEMPRO hESC SFM or a culture supernatant of MEF as the maintenance culture broth, as described in Example 3. The human ES cells were single dissociated as reported previously (Nat Biotechnol 2007; 25:681-686), seeded to a 24-well plate (coated with an extracellular matrix from human decidua-derived mesenchymal cells) at 2000 cells per well, and cultured for 7 days. During the first 2 days, the cells were cultured in a culture broth supplemented with the ROCK inhibitor Y-27632 (10 μM; Mol Pharmacol 2000; 57:976-983).

### (Results)

The cells cultured on the extracellular matrix from human decidua-derived mesenchymal cells using STEMPRO hESC SFM or a culture supernatant of MEF increased their cell count 65 folds and 25 folds, respectively, 7 days later. These cells colonized; the colonies were strongly positive for the undifferentiation marker alkaline phosphatase.

### Example 6

### Maintenance Culture of Human iPS Cells on Extracellular Matrix from Human Decidua-Derived Mesenchymal Cells (Methods)

Human iPS cells (253G4; Nat Biotechnol 2008; 26:101-106) were subjected to passage culture in the form of cell masses using an extracellular matrix from human decidua-derived mesenchymal cells and STEMPRO hESC SFM or a culture supernatant of MEF as the maintenance culture broth, as described in Example 3. The cells were seeded to a 6-well plate (coated with an extracellular matrix from human decidua-derived mesenchymal cells) at 33,000 cells per well, and subjected to passage maintenance culture for 16 days.

### (Results)

During the maintenance culture on the extracellular matrix from human decidua-derived mesenchymal cells using STEMPRO hESC SFM or a culture supernatant of MEF, the human iPS cells proliferated well and increased their cell count 200 folds and 60 folds, respectively, over the 16 days. The cells were strongly positive for the undifferentiation markers Oct3/4, SSEA3, TRA-1-60, alkaline phosphatase and the like. These findings show that human iPS cells, like human ES cells, can be maintenance-cultured using a chemically synthetic medium and an extracellular matrix from human decidua-derived mesenchymal cells.

### INDUSTRIAL APPLICABILITY

The present invention makes it possible to perform maintenance culture of human pluripotent stem cells safely and efficiently. Because the culture agent and container for pluripotent stem cells of the present invention afford sufficient quantitative supplies and allow preparation of a large amount thereof at one time, uniform quality control (activity and safety) is possible. The container of the present invention permits long-term storage in a refrigerator; in preparation for clinical application, the container may be previously subjected to adequate testing on safety and activity to ensure controlled quality.

This application is based on patent application Nos. 2008-328339 (filing date: Dec. 24, 2008) and 2009-197239 (filing date: Aug. 27, 2009), filed in Japan, the contents of which are incorporated in full herein by this reference.

1. A method of culturing pluripotent stem cells in the presence of decidua-derived cells or an extracellular matrix derived from the cell.

2. The method according to claim 1, wherein the decidua-derived cell is a mesenchymal cell.

3. The method according to claim 1, wherein both the decidua-derived cell and the pluripotent stem cells are derived from a mammal of the same species.

4. The method according to claim 3, wherein the mammal is a human.

5. The method according to claim 1, wherein the pluripotent stem cells are human ES cells or human iPS cells.

6. A culture agent for pluripotent stem cells, containing decidua-derived cells or an extracellular matrix derived from the cell.

7. The agent according to claim 6, wherein the decidua-derived cell is a mesenchymal cell.

8. The agent according to claim 6, wherein both the decidua-derived cell and the pluripotent stem cells are derived from a mammal of the same species.

9. The agent according to claim 8, wherein the mammal is a human.

10. The agent according to claim 6, wherein the pluripotent stem cells are human ES cells or human iPS cells.

11. A container for culturing pluripotent stem cells, coated with an extracellular matrix from decidua-derived cells.

12. The container according to claim 11, wherein the decidua-derived cell is a mesenchymal cell.

13. The container according to claim 11, wherein both the decidua-derived cell and the pluripotent stem cells are derived from a mammal of the same species.

14. The container according to claim 13, wherein the mammal is a human.

15. The container according to claim 11, wherein the pluripotent stem cells are human ES cells or human iPS cells.

16. A kit for culturing pluripotent stem cells, comprising (i) and (ii) below:
(i) a culture agent for pluripotent stem cells, comprising decidua-derived cells or an extracellular matrix derived from the cell; and
(ii) an explanatory document stating that the kit should be used, or can be used, for culturing pluripotent stem cells.

17. A kit for culturing pluripotent stem cells, comprising (i) and (ii) below:
(i) a container for culturing pluripotent stem cells, coated with an extracellular matrix from decidua-derived cells; and
(ii) an explanatory document stating that the kit should be used, or can be used, for culturing pluripotent stem cells.
